(19) United States
(12) Patent Application Publication
(10) Pub. No.: US 2023/0357825 A1
(43) Pub. Date: Nov. 9, 2023

(54) CELL PRESERVATION REAGENTS AND METHODS OF USE

(71) Applicant: Truckee Applied Genomics, LLC, Reno, NV (US)

(72) Inventor: Tony K. Baker, Truckee, CA (US)

(21) Appl. No.: 18/150,988

(22) Filed: Jan. 6, 2023

Related U.S. Application Data
(63) Continuation of application No. PCT/US2021/040539, filed on Jul. 6, 2021.
(60) Provisional application No. 63/049,074, filed on Jul. 7, 2020.
(60) Provisional application No. 63/084,502, filed on Sep. 28, 2020.

(51) Int. Cl. C12Q 1/6806; A01N 1/02

(52) U.S. Cl. CPC C12Q 1/6806; A01N 1/0226

(57) ABSTRACT

Reagents and methods of use thereof for stabilizing a biological sample, including preserving or preventing destruction of a biological molecule within the biological sample, are provided.

### CROSS-REFERENCE

This application is a continuation application of International Application No. PCT/US2021/040539, filed Jul. 6, 2021, which claims the benefit of U.S. Provisional Patent Application No. 63/049,074, filed Jul. 7, 2020, and U.S. Provisional Patent Application No. 63/084,502, filed Sep. 28, 2020, each of which is incorporated by reference in its entirety.

### BACKGROUND

After collection, biological samples can be stored and/or processed prior to analysis. Conditions during storage and processing can affect the integrity of a biological sample and its molecular components, such as deoxyribonucleic acid (DNA), ribonucleic acid (RNA), and protein. Preservation of the integrity of such molecular components can influence the results of their analysis, and can be important for improvement of sensitivity and/or sensitivity of analytical methods.

### SUMMARY

In an aspect, provided herein is a method for stabilizing a biological sample, the method comprising: (a) providing a reagent comprising a kosmotrope, a chaotrope, and a viral inactivating agent, the reagent configured to protect a biological molecule in the biological sample from destabilization or degradation; and (b) contacting the biological sample comprising the biological molecule with the reagent to yield a stabilized biological sample comprising the biological molecule. In some embodiments, the biological sample is obtained from a subject. In some embodiments, the biological sample comprises a sample of a component of a respiratory system. In some embodiments, the biological sample comprises a sample obtained via a nasopharyngeal swab. In some embodiments, the biological molecule comprises a protein, a deoxyribonucleic acid (DNA) molecule, a ribonucleic acid (RNA) molecule, or any combination or variant thereof. In some embodiments, the biological sample is of a subject having or suspected of being infected with a pathogen. In some embodiments, the pathogen is severe acute respiratory syndrome coronavirus-19 (SARS-CoV-2). In some embodiments, the biological sample comprises SARS-CoV-2. In some embodiments, the biological sample comprises a ribonucleic acid (RNA) of SARS-CoV-2. In some embodiments, the biological sample comprises an RNA fragment of SARS-CoV-2. In some embodiments, the RNA is of SARS-CoV-2.

In some embodiments, the kosmotrope comprises at least a first kosmotrope, and a second kosmotrope different from the first kosmotrope. In some embodiments, the kosmotrope comprises glycerol, α,α-trehalose, or a combination thereof. In some embodiments, the chaotrope is sodium thiocyanate. In some embodiments, the reagent further comprises a chelator. In some embodiments, the chelator is ethylenediaminetetraacetic acid (EDTA). In some embodiments, the reagent further comprises dimethylsulfoxide (DMSO). In some embodiments, the reagent does not comprise DMSO. In some embodiments, the reagent does not comprise a fixing agent. In some embodiments, the reagent further comprises a sodium phosphate buffer. In some embodiments, the reagent further comprises a potassium phosphate buffer. In some embodiments, the reagent further comprises a monobasic potassium phosphate buffer, a tribasic potassium phosphate buffer, or a combination thereof.

In some embodiments, the viral-inactivating agent is a viral inactivation viricide. In some embodiments, the viral-inactivating agent comprises a solvent, a detergent, a surfactant, or any combination or variant thereof. In some embodiments, the viral-inactivating agent is a pharmaceutically acceptable excipient. In some embodiments, the viral-inactivating agent comprises a nonionic surfactant. In some embodiments, the viral-inactivating agent comprises an octylphenol ethoxylate (OPE) or a nonylphenol ethoxylate (NPE). In some embodiments, the viral-inactivating agent is non-toxic to humans at the concentrations used.

In some embodiments, the biological molecule in the stabilized biological sample has a concentration of about 10 genetic equivalent copies per microliter (GEC/μL) or less. In some embodiments, the biological molecule in the stabilized biological sample is detected at about 16 genetic equivalent copies per microliter (GEC/μL) or less. In some embodiments, the biological molecule in the stabilized biological sample is detected about 8 GEC/μL or less. In some embodiments, the biological molecule in the stabilized biological sample is detected about 7 GEC/μL. In some embodiments, the biological molecule in the stabilized biological sample is detectable by polymerase chain reaction in less than 40 cycles after about 28 days or more. In some embodiments, the biological molecule in the stabilized biological sample is detectable by polymerase chain reaction after about 28 days or more without refrigeration.

In some embodiments, the reagent inactivates a pathogen. In some embodiments, the reagent inactivates the pathogen within about 30 seconds. In some embodiments, the reagent inactivates the pathogen within about 10 seconds. In some embodiments, the reagent inactivates the pathogen within about 10 seconds without damaging the genetic material of the pathogen.

In some embodiments, the reagent further comprises a biocide. In some embodiments, the biocide has broad spectrum antimicrobial activity. In some embodiments, the biocide comprises an organic compound. In some embodiments, the reagent further comprises one or more substituted or unsubstituted isothiazolinones. In some embodiments, the reagent further comprises 5-Chloro-2-methyl-3(2H)-isothiazolone (CMIT), 2-methyl-3(2H)-isothiazolone (MIT), or a combination thereof.

In some embodiments, the reagent enhances stability to degradation of genetic material following exposure of the biological sample or the stabilized biological sample to high temperatures. In some embodiments, the reagent enhances stability to degradation of genetic material following exposure of the biological sample or the stabilized biological sample to one or more freeze/thaw cycles. In some embodiments, the reagent enhances stability to enzymatic degradation of genetic material. In some embodiments, the enzymatic degradation comprises degradation by a metalloprotease, endonuclease, exonuclease, ribonuclease, or a combination thereof.

In another aspect, provided herein is a reagent for stabilizing a biological sample, comprising: (a) a kosmotrope, and a chaotrope for protecting a biological molecule in the biological sample from destabilization or degradation; and (b) a viral-inactivating agent for inactivating a virus in the biological sample within 30 seconds. In some embodiments, the reagent is configured to inactivate the virus in the biological sample within 10 seconds. In some embodiments, the reagent further comprises a biocide. In some embodiments, the reagent further comprises one or more substituted or unsubstituted isothiazolinones. In some embodiments, the reagent stabilizes the biological molecule from the destabilization or degradation, such that the biological molecule is detectable at the concentration of about 10 genetic equivalent copies per microliter (GEC/μL) or less. In some embodiments, the biological molecule is detectable for about 28 days or more. In some embodiments, the biological molecule is detectable without refrigeration.

In some embodiments, the reagent further comprises dimethylsulfoxide (DMSO). In some embodiments, the reagent further comprises a chelator. In some embodiments, the reagent further comprises ethylenediaminetetraacetic acid (EDTA). In some embodiments, the kosmotrope comprises at least a first kosmotrope and a second kosmotrope, wherein the second kosmotrope is different from the first kosmotrope. In some embodiments, the kosmotrope comprises glycerol, α,α-trehalose, or a combination thereof. In some embodiments, the reagent further comprises sodium thiocyanate. In some embodiments, the reagent further comprises a monobasic potassium phosphate buffer, a tribasic potassium phosphate buffer, or a combination thereof. In some embodiments, the reagent further comprises an octylphenol ethoxylate (OPE) or a nonylphenol ethoxylate (NPE).

In some embodiments, the biological molecule is a ribonucleic acid (RNA). In some embodiments, the biological molecule is a viral RNA. In some embodiments, the biological molecule is SARS-CoV-2 RNA.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed. Reagents herein can act to stabilize biological samples, such as maintaining cellular viral RNA integrity of both intracellular and extracellular substances and structures, enzyme activity, protein structures, or other metabolic functions. In some cases, reagents herein can mediate pH and thermal stress in samples. In some embodiments, reagents herein can serve as SARS-CoV-2 buffer reagents for collection, storage, or transportation of samples to be used in laboratory tests, for example polymerase chain reaction (PCR) or reverse transcription polymerase chain reaction (rtPCR). In some embodiments, reagents herein can serve as a viral transport medium (VTM). Biological samples stabilized by reagents herein can provide better RNA sequence stabilization yielding lower Levels of Detection (LOD)/improved test sensitivity, and fewer false positives and false negative test results, for example, when compared with a same biological sample not stabilized by reagents or a same biological sample stabilized by another available reagent (e.g., formalin). In some instances, a reagent (e.g., a VTM) may stabilize or slow the degradation of a biological sample, a viral particle or fragments thereof (e.g., viral proteins or protein fragments, genetic information such as DNA or RNA, or other identifiable biomarkers). A VTM may increase the amount of time that a biological sample can be stored or transported while still maintaining information (e.g., genetic or protein sequence information) about the biological sample (e.g., presence, absence, or identity of a viral particle or fragment thereof contained in the sample or particle). As used herein, “half-life” may refer to the amount of time it takes for half of the information (e.g., genetic, peptidic) to be degraded within a biological sample after acquisition from a subject. In some instances, a VTM increases the half-life of a biological sample or a viral particle (and the information disposed therein) by up to about 10%, about 20%, about 30%, about 40%, about 50%, about 75%, about 100%, about 200%, about 300%, about 400%, about 500%, about 1000%, or more, as compared to a biological sample or viral particle stored or transported in saline or another non-VTM medium.

Reagents herein can serve as a non-toxic replacement, and in some cases direct replacement, for fixing agents such as formalin in processing a biological sample. In some embodiments, reagents herein require no pathology workflow changes from an analogous established pathology workflow using a fixing agent. Reagents herein can provide equivalent, essentially equivalent, or improved results in analyses of biological samples compared with a same biological sample not stabilized by a reagent or a same biological sample stabilized by another available reagent, such as a reagent comprising a fixing agent. For example, reagents herein can provide equivalent, essentially equivalent, or improved results in anatomical pathology tests or current molecular pathology tests, or emerging molecular pathology tests.

In some embodiments, reagents herein can serve to stabilize or preserve biological samples of subjects suspected of being infected with a pathogen (e.g., a virus) such as the severe acute respiratory syndrome coronavirus-2 (SARS-CoV-2) virus, which can be causative for coronavirus-19 disease (COVID-19). Such samples stabilized or preserved using reagents provided herein can be analyzed for research, diagnostic, or other purposes.

Metabolically intact cells can be more diagnostically useful than cells that are not metabolically intact. Cellular components including proteins, surface antigens, DNA, RNA, mRNA and regulatory enzymes, which can be destroyed or significantly degraded by environmental or other factors can be protected by reagents provided herein, rendering these cellular components metabolically intact.

Sample integrity and stability are the key to successful analysis, including SARS-CoV-2 testing. Reagents provided herein can provide or encourage sample integrity and stability across a broad spectrum of pathology uses.

### Reagents

Provided herein are reagents. A reagent can be a preservation reagent, such as a biological sample preservation reagent, a cell preservation reagent, a tissue preservation reagent, a cancer preservation reagent, or a biomolecule preservation reagent (i.e., a protein preservation reagent, a DNA preservation reagents, or an RNA preservation reagent). A preservation reagent can preserve a cell or tissue. In some cases, a preservation reagent can preserve a cell or tissue of a subject, for example a subject suspected of having a disease or condition. In some embodiments, a preservation reagent can stabilize a single cell or a plurality of cells.

Reagents provided herein can be non-toxic or non-biohazardous. In some embodiments, reagents provided herein do not require special or extensive disposal protocols. In some embodiments components of reagents provided herein can be present at a low or very low molar concentration while accomplishing the final endpoint of a stabilized biological sample or component thereof, for example antigenic sequences or RNA sequences for analysis of biological samples, such as clinical analysis of SARS-CoV-2 biological samples (e.g., a sample comprising SARS-CoV-2 or a sample of a subject suspected of being infected with SARS-CoV-2). Reagents herein are also capable of stabilizing pathogen samples that contain cells, fragments of lysed cells, virus material, fungal material, bacterial material, or combinations or fragments thereof. In some embodiments, the reagents described herein do not produce cyanide gas under prescribed use conditions (e.g., laboratory conditions). In some embodiments, reagents described herein produce no or immeasurable amounts of cyanide gas. In some embodiments, the amount of cyanide gas produced by use of a reagent described herein is less than 50%, less than 40%, less than 30%, less than 25%, less than 20%, less than 15%, less than 10%, less than 5%, less than 4%, less than 3%, less than 2%, or less than 1% of the amount produced by other molecular transport media. In some embodiments, a reagent described herein does not produce cyanide gas during polymerase chain reaction (PCR) or any variant genetic amplification technique known in the art.

A reagent can be a stimulating reagent, for example a gene expression stimulating reagent, a protein expression stimulating reagent, or a cancer stimulating reagent. In some cases, a stimulating reagent can stimulate growth or a process. For example, in some cases, a cancer stimulating reagent can stimulate or increase the growth of cancer cells or tissue, a gene expression stimulating reagent can stimulate the expression of genes (e.g., translation of DNA into RNA), and a protein expression stimulating reagent can stimulate the expression of protein (e.g., transcription of RNA into protein).

A reagent can maintain cellular viability of a biological sample. In some embodiments, a reagent can maintain viability or integrity of one or more molecular components of a biological sample, such as RNA, DNA, or protein.

A biological sample herein can comprise a biomolecule, such as RNA, DNA, or protein. RNA can be, for example, viral RNA, tRNA, rRNA, mRNA, or siRNA. DNA can be, for example, chromosomal DNA, mitochondrial DNA, viral DNA, circular DNA (e.g., a plasmid or bacterial DNA), or synthetic DNA. A protein can be, for example, a hormone, a metabolic protein, a structural protein, a transporter protein, a GPCR, a prion, a secreted protein, or another type of protein.

A biological sample can be a biological fluid (e.g., urine, blood, cerebrospinal fluid, plasma, semen, vaginal discharge, sweat, saliva, or other fluid), bone marrow, a cell (e.g., a white blood cell, a primary tumor cell, a metastatic tumor cell, a cultured cell, a skin cell, an epithelial cell, or another type of cell). A biological sample can comprise a biological sample of a component of a respiratory system of a subject. For example, a biological sample can comprise a mucus sample, a nasal swab, a sample obtained via a nasopharyngeal swab, a sample obtained via bronchoalveolar lavage, or another sample originating from or relating to a respiratory system component.

In some embodiments, a biological sample can comprise a pathogen, such as a virus, bacteria, fungus, yeast, parasite, or other type of pathogen. In some embodiments, a biological sample can be altered by such a pathogen, such as having altered expression of one or more genes or proteins, or altered metabolism. In some embodiments, a biological sample can comprise components of a pathogen, such as RNA, DNA, or protein of the pathogen. In some embodiments, for example, a biological sample can comprise a severe acute respiratory syndrome coronavirus-2 (SARS-CoV-2) virus or a component thereof (e.g., RNA, DNA, or protein).

In some cases, a biological sample can comprise cancer. Cancer can refer to or describe the physiological condition in mammals that is typically characterized by unregulated cell growth. Examples of cancer can include, but are not limited to, breast cancer, ovarian cancer, colon cancer, lung cancer, prostate cancer, hepatocellular cancer, gastric cancer, pancreatic cancer, cervical cancer, uterine cancer, stomach cancer, liver cancer, bladder cancer, testicular cancer, retinal cancer, cancer of the urinary tract, thyroid cancer, renal cancer, carcinoma, melanoma, myeloma, lymphoma, osteosarcoma, and brain cancer.

A cancerous biological sample can comprise a tumor. In some embodiments, a tumor can be a neoplastic cell growth and/or proliferation of tissue, whether malignant or benign. In some cases, a tumor can include pre-cancerous or cancerous cells or tissues.

A biological sample can include a tissue section such as a biopsy or autopsy sample. A biological sample can be obtained from a eukaryotic organism, for example a mammal. In some cases, a biological sample can be obtained from a primate (e.g., chimpanzee or human), cow, dog, cat, rodent (e.g., guinea pig, rat, or mouse), rabbit, or a bird, reptile, or fish.

A biopsy can refer to a process comprising removal of a tissue sample for diagnostic or prognostic evaluation. In some cases, a biopsy can refer to a tissue specimen obtained by a process of removal for diagnostic or prognostic evaluation. Any biopsy technique known in the art can be applied to the diagnostic and prognostic methods of the present invention. The biopsy technique applied can depend on the tissue type to be evaluated (e.g., lung, bone marrow, liver, skin, bone, etc.), the size of the tumor, the type of the tumor, or the location of the tumor, among other factors. Representative biopsy techniques can include, but are not limited to, excisional biopsy, incisional biopsy, needle biopsy, surgical biopsy, and bone marrow biopsy. An excisional biopsy can refer to the removal of a tumor mass, typically an entire tumor mass, in some cases with a small margin of normal tissue surrounding it. An incisional biopsy can refer to the removal of a wedge of tissue from within a tumor or tissue. In some cases, a diagnosis or prognosis made by endoscopy or radiographic guidance can include a “core-needle biopsy” or a “fine-needle aspiration biopsy” which can result in the obtention of a sample that can comprise a suspension of cells from within a target tissue.

In some cases, a reagent can enable the analysis of genetic expression (e.g., RNA analysis) in a sample. Analysis of genetic expression can refer to analyses that deal with detecting the over expression, under expression or differentially expressed genes of a cell, particularly in cancer cells.

Such reagents can for example allow the testing of accurate gene expression of viable cells, such as cancer cells. In some cases, the testing of gene expression can include polymerase chain reaction (PCR) analysis (e.g., standard PCR, reverse transcriptase PCR or real time PCR), northern blotting, in situ hybridization, expression microarrays, or sequencing analysis (e.g., RNAseq). In some embodiments, reagents provided herein enable genetic analysis of a pathogen, e.g., a virus, a fungus, a bacterium, an invasive or hyperproliferating eukaryotic cell, and the like. For example, a reagent herein may stabilize genetic material without disrupting cells or proteins in the sample, thereby enhancing the accuracy of genetic analysis (e.g., nucleic acid sequencing, quantification, detection, identification, etc.). In other embodiments, a reagent stabilizes genetic material while also disrupting a pathogen's cellular or protein structure, thereby inactivating the pathogen while enabling its genetic analysis. In some embodiments, a reagent herein stabilizes the genetic material of a pathogen for analysis for up to 5 days, 6 days, 7 days, 8 days, 9 days, 10 days, 11 days, 12 days, 13 days, 14 days, 21 days, 28 days, 35 days, or longer, wherein the genetic material can still be analyzed after such storage. In some embodiments, a reagent provided herein stabilizes a pathogen's genetic material for up to 28 days. In some embodiments, a reagent provided herein stabilizes a pathogen's genetic material for 28 days or more. In some embodiments, a reagent provided herein stabilizes a pathogen's genetic material for at least 28 days. In some embodiments, a reagent provided herein stabilizes a pathogen's genetic material for 42 days or more.

In some embodiments, a reagent provided herein enables genetic analysis of a pathogen after 10 days, after 14 days, after 21 days, after 28 days, after 35 days, after 42 days, or more of storage or transport without refrigeration. In some embodiments, a reagent provided herein enables genetic analysis of a pathogen after 14 days of storage or transport without refrigeration. In some embodiments, a reagent provided herein enables genetic analysis of a pathogen after 21 days of storage or transport without refrigeration. In some embodiments, a reagent provided herein enables genetic analysis of a pathogen after 28 days of storage or transport without refrigeration. In some embodiments, a reagent provided herein enables genetic analysis of a pathogen after 35 days of storage or transport without refrigeration. In some embodiments, a reagent provided herein enables genetic analysis of a pathogen after 42 days of storage or transport without refrigeration. In some embodiments, as described herein, a reagent preserves genetic information of a sample (e.g., a pathogen) for storage and/or transport of specimens without refrigeration, e.g., at ambient temperature (typically 20-25° C.) for up to and including about 28 days. In some embodiments, as described herein, a reagent preserves genetic information of a sample (e.g., a pathogen) for storage and/or transport of specimens without refrigeration, e.g., at ambient temperature (typically 20-25° C.) for about 28 days or more. In some embodiments, a reagent herein preserves and/or protects the nucleic acid structure and/or sequence of a biological sample (e.g., of a pathogen such as a virus) from changes in temperature. For example, in some embodiments, a reagent herein protects genetic material from exposure to high temperature. In other embodiments, a reagent herein protects genetic material from exposure to one or more freeze/thaw cycles.

“High temperature” as used herein can be temperature that is greater than ambient temperature, e.g., about 25+/−5° C. In some embodiments, high temperature is equal to or greater than about 30° C., about 35° C., about 40° C., about 45° C., about 50° C., about 55° C., about 60° C., or more. In some embodiments, high temperature is about 35° C. to about 55° C. In other embodiments, high temperature is about 40° C. to about 50° C. In some embodiments, a reagent herein protects genetic information from thermal degradation. In some embodiments, a reagent herein protects genetic information from thermal degradation that may occur from brief exposure (e.g., up to about 5 minutes, about 10 minutes, about 15 minutes, about 20 minutes, about 30 minutes, about 45 minutes, or up to about 60 minutes) to high temperature. In some embodiments, a reagent herein protects genetic information from thermal degradation that may occur from prolonged exposure (e.g., up to about 2 hours, about 3 hours, about 4 hours, about 5 hours, about 6 hours, about 12 hours, about 24 hours, about 48 hours, about 3 days, about 4 days, about 5 days, about 10 days, about 14 days, about 21 days, about 28 days, or more) to high temperature.

In some embodiments, a reagent herein protects genetic information from thermal degradation that may occur from intermittent or variable exposure to high temperature, such as may occur during transportation or storage without refrigeration. In some embodiments, a reagent herein protects genetic information from thermal degradation that may occur from temperature changes of about 10° C., about 15° C., about 20° C., about 25° C., about 30° C., about 40° C., about 50° C., about 60° C., about 70° C., or more. For example, a reagent herein may prevent thermal degradation of genetic material that may otherwise occur during one or more freeze/thaw or hot/cold cycles. A hot/cold cycle includes any change in temperature disclosed above. In some examples, a reagent prevents thermal degradation of genetic material from temperature changes of about 25° C., about 30° C., about 40° C., or about 50° C. In some embodiments, a reagent herein prevents thermal degradation at temperatures up to about 30° C., about 35° C., about 40° C., about 45° C., about 50° C., about 55° C., or greater. In some embodiments, a reagent herein prevents thermal degradation at temperatures up to about 40° C. In some embodiments, a reagent herein prevents thermal degradation at temperatures up to about 45° C. In some embodiments, a reagent herein prevents thermal degradation at temperatures up to about 50° C. In some embodiments, a reagent herein prevents thermal degradation at temperatures up to about 55° C.

In some embodiments, a reagent provided herein enables genetic analysis of a pathogen after 10 days, 14 days, after 21 days, after 28 days, after 35 days, after 42 days, or more of storage or transport under refrigerated conditions. In some embodiments, a reagent provided herein enables genetic analysis of a pathogen after 1 month, 2 months, 3 months, 6 months, 12 months, 18 months, or more under refrigerated conditions. In some embodiments, a reagent provided herein enables genetic analysis of a pathogen after 1 year, 2 years, 3 years, 5 years, 10 years, or more under refrigerated conditions. In some embodiments, a reagent provided herein enables genetic analysis of a pathogen indefinitely under refrigerated conditions.

As used herein, “refrigerated conditions” or “refrigeration” comprises any conditions wherein temperature (and optionally humidity) are controlled within about +/−5 degrees Celsius at a temperature that is about 20° C. or less. In some embodiments, refrigerated conditions refer to a controlled temperature that is about 10° C. or less. In some embodiments, refrigerated conditions refer to a controlled temperature that is about 5° C. or less. In some embodiments, refrigerated conditions refer to a controlled temperature that is about 0° C. or less. In some embodiments, refrigerated conditions refer to a controlled temperature that is about −10° C. or less. In some embodiments, refrigerated conditions refer to a controlled temperature that is about −20° C. or less. In some embodiments, refrigerated conditions refer to a controlled temperature that is about −40° C. or less. In some embodiments, refrigerated conditions refer to a controlled temperature that is about −80° C. or less.

In some embodiments, a reagent herein enables genetic analysis (e.g., detection, quantification, sequencing, identification, and the like) of genetic information (e.g., RNA, DNA) at low levels. In some embodiments, “low levels” constitutes a lower concentration or quantity relative to the current standard. In some embodiments, low levels are defined in terms of genomic equivalent copies (GEC) per milliliter (mL); (GEC/mL). In some embodiments, low levels are defined in terms of genomic equivalent copies (GEC) per microliter (μL); (GEC/μL). In some embodiments, a reagent herein enables genetic analysis of a pathogen (e.g., of an inactivated virus) at lower levels than other FDA-approved viral transport media. In some embodiments, a reagent herein enables genetic analysis of a pathogen (e.g., of an inactivated virus) at lower levels than a reference reagent. In some embodiments, a reference reagent is a DNA/RNA Shield™ reagent, device, or kit (Zymo Research). In some embodiments, a reference reagent is a CDC Viral Transport Medium (VTM) reagent, device, or kit (Center for Disease Control and Prevention). In some embodiments, a reagent herein enables genetic analysis of genetic information at lower levels (in GEC/μL) than achievable with a reference reagent (e.g., a DNA/RNA Shield™ or CDC VTM reagent, device, or kit).

In some embodiments, a reagent herein enables genetic analysis of a pathogen at as little as about 1000 GEC/μL, about 500 GEC/μL, 100 GEC/μL, about 50 GEC/μL, about 25 GEC/μL, about 20 GEC/μL, about 15 GEC/μL, about 10 GEC/μL, about 5 GEC/μL, or less. In some embodiments, a reagent herein enables genetic analysis of a pathogen in concentrations of about 32 GEC/μL, 16 GEC/μL, 8 GEC/μL, 4 GEC/μL, or less. In some embodiments, a pathogen's genetic material is detected at concentrations of about 4 GEC/μL to about 8 GEC/μL. In some embodiments, a pathogen's genetic material is detected at concentrations of about 8 GEC/μL to about 16 GEC/μL. In some embodiments, a pathogen's genetic material is detected at concentrations of about 7 GEC/μL to about 500 GEC/μL. In some embodiments, a pathogen's genetic material is detected at concentrations of about 16 GEC/μL. In some embodiments, a pathogen's genetic material is detected at concentrations of about 12 GEC/μL. In some embodiments, a pathogen's genetic material is detected at concentrations of about 8 GEC/μL or less. In some embodiments, a pathogen's genetic material is detected at concentrations of about 7 GEC/μL or less. In some embodiments, a pathogen's genetic material is detected at concentrations of about 6 GEC/μL or less. In some embodiments, a pathogen's genetic material is detected at concentrations of about 5 GEC/μL or less. In some embodiments, a pathogen's genetic material is detected at concentrations of about 4 GEC/μL or less.

Analysis of genetic expression can identify the differential expression, overexpression or underexpression of particular genes. “Overexpress,” “overexpression,” or “overexpressed” can interchangeably refer to a protein or nucleic acid (RNA) that is translated or transcribed at a detectably greater level, usually in a cancer cell, in comparison to a normal cell. This can include overexpression due to transcription, post transcriptional processing, translation, post-translational processing, cellular localization (e.g., organelle, cytoplasm, nucleus, cell surface), and RNA and protein stability, as compared to a normal cell. Overexpression can be detected using conventional techniques for detecting mRNA (i.e., RT-PCR (reverse transcriptase-PCR), PCR, hybridization) or proteins (i.e., ELISA, immunohistochemical techniques). PCR can be a technology where a nucleotide is amplified via temperature cycling techniques in the presence of a nucleotide polymerase, preferably a DNA polymerase. Overexpression can be 10%, 20%, 30%, 40%, 50%, 60%, 70%, 80%, 90% or more in comparison to a normal cell. In certain instances, overexpression is 1-fold, 2-fold, 3-fold, 4-fold, or higher levels of transcription or translation in comparison to a normal cell.

“Underexpress,” “underexpression,” or “underexpressed” or “downregulated” can interchangeably refer to a protein or nucleic acid that is translated or transcribed at a detectably lower level in a cancer cell, in comparison to a normal cell. The term includes underexpression due to transcription, post transcriptional processing, translation, post-translational processing, cellular localization (e.g., organelle, cytoplasm, nucleus, cell surface), and RNA and protein stability, as compared to a control. Underexpression can be detected using conventional techniques for detecting mRNA (i.e., RT-PCR, PCR, hybridization) or proteins (i.e., ELISA, immunohistochemical techniques). Underexpression can be 10%, 20%, 30%, 40%, 50%, 60%, 70%, 80%, 90% or less in comparison to a control. In certain instances, underexpression is 1-fold, 2-fold, 3-fold, 4-fold, or lower levels of transcription or translation in comparison to a control.

“Differentially expressed” or “differentially regulated” refers generally to a protein or nucleic acid that can be overexpressed (upregulated) or underexpressed (downregulated) in one sample compared to at least one other sample, for example in a cancer patient compared to a sample of non-cancerous tissue in the context of the present invention.

A reagent can comprise a sulfone. In some cases, the sulfone can be polyvinyl sulfone. In some cases, the sulfone can be a derivative of polyvinyl sulfone.

In some cases, a reagent can comprise a chaotrope. A chaotrope can be a compound that can interact weakly with water molecules. In some cases, a chaotrope can disrupt a water molecule hydrogen bonded network around protein molecules. Examples of chaotropes can include, without limitation, SCN⁻(sodium thiocyanate), H₂PO₄⁻, HCO₃⁻, I⁻, Cl⁻, NO₃⁻, NH₄⁺, Cs⁺, K⁺, (NH₂)₃C⁺, guanidinium, any salt of guanidinium, Br⁻, or Rb⁺. Such compounds can have effects on water distribution around a cell and help in maintaining cell viability.

In some embodiments, a chaotrope can have a final concentration in the reagent of ranging from about 0.1 Molar to about 2 Molar. In some cases, the final concentration of a chaotrope can be at least about 1 mM, at least about 10 mM, at least 0.05 Molar, at least about 0.1 Molar at least 0.5 Molar at least about 1 Molar, at least about 1.75 Molar, or at least about 2 Molar. In some cases, to a maximum of at most about 1 Molar, at most about 2 Molar, at most about 3 Molar, at most about 4 Molar, or at most about 5 Molar.

In some cases, a reagent can comprise a kosmotrope. A kosmotrope can be a compound that can interact strongly with water molecules. In some cases, a kosmotrope can organize water molecules in around protein molecules (e.g., in a favorable manner). A biomaterial stabilizing composition may include a kosmotrope in some embodiments. Without being limited to any specific mechanism(s) of action, a kosmotrope can stabilize and/or improve water-water interactions in an aqueous composition. Examples of kosmotropes can include, without limitation, glycerol, proline (e.g., L-proline), trehalose (e.g., D-(+) trehalose, D-(+) trehalose dihydrate), α,α-Trehalose, glycine betaine, glucose, dextrose, glutamic acid, and/or aspartic acid. Examples of a kosmotrope, in some embodiments, can include SO₄⁻, HPO₄⁻, Ca₂⁺, Mg₂⁺, Li⁺, Na⁺, OH⁻, and/or PO₄²⁻.

A kosmotrope can have a final concentration in the reagent of at least about 0.1 mM, at least about 1 mL, at least about 10 mM, at least about 0.1 Molar, at least about 1 Molar, at least about 1.5 Molar, or at least about 2.0 Molar. A kosmotrope can have a final concentration in a reagent of at most about 1.0 Molar, at most about 1.5 Molar, to about 2 Molar, at most about 3 Molar, at most about 4 Molar, or at most about 5 Molar.

In some cases, a reagent can comprise a buffer. A buffer can be a compound which gives a mixture a desired pH. For example, in some cases, a desired pH can be from about 4.5 to about 8.5. In some cases, a desired pH can be about 4.5, about 5.0, about 5.5, about 6.0, about 6.5, about 7.0, about 7.5, about 8.0, or about 8.5. In some cases, a desired pH can be between about 4.5 and about 8.5, between about 4.5 and about 8.0, between about 4.5 and about 7.5, between about 4.5 and about 7.0, between about 4.5 and about 6.5, between about 4.5 and about 6.0, between about 4.5 and about 5.5, between about 4.5 and about 5.0, between about 5.0 and about 8.5, between about 5.0 and about 8.0, between about 5.0 and about 7.5, between about 5.0 and about 7.0, between about 5.0 and about 6.5, between about 5.0 and about 6.0, between about 5.0 and about 5.5, between about 5.5 and about 8.5, between about 5.5 and about 8.0, between about 5.5 and about 7.5, between about 5.5 and about 7.0, between about 5.5 and about 6.5, between about 5.5 and about 6.0, between about 6.0 and about 8.5, between about 6.0 and about 8.0, between about 6.0 and about 7.5, between about 6.0 and about 7.0, between about 6.0 and about 6.5, between about 6.5 and about 8.5, between about 6.5 and about 8.0, between about 6.5 and about 7.5, between about 6.5 and about 7.0, between about 7.0 and about 8.5, between about 7.0 and about 8.0, between about 7.0 and about 7.5, between about 7.5 and about 8.5, between about 7.5 and about 8.0, or between about 8.0 and about 8.5. A desired pH can be strongly acidic, weakly acidic, neutral, weakly basic, or strongly basic.

In some embodiments, a suitable buffer may be selected from Good buffers (e.g., HEPES), potassium acetate, sodium phosphate, potassium bicarbonate, tris(hydroxyamino)methane (Tris), and combinations thereof. For example, a buffer may include potassium acetate, sodium acetate, potassium phosphate (monobasic, dibasic, tribasic), sodium phosphate, Tris, N-(2-hydroxyethyl)piperazine-N′-(2-ethanesulfonic acid) (HEPES) buffer, 3-(N-morpholino)propane sulfonic acid (MOPS) buffer, 2-[(2-amino-2-oxoethyl)amino]ethanesulfonic acid (ACES) buffer, N-(2-acetamido)-2-iminodiacetic acid buffer (ADA), 3-[(1,1-dimethyl-2-hydroxyethyl)amino]-2-propanesulfonic acid (AMPSO) buffer, N,N-bis(2-hydroxyethyl)-2-aminoethanesulfonic acid (BES) buffer, Bicine (N,N-bis(2-hydroxyethylglycine) buffer, bis-(2-hydroxyethyl)imino-tris(hydroxymethyl)methane (Bis-Tris) buffer, 3-(cyclohexylamino)-1-propanesulfonic acid (CAPS) buffer, 3-(cyclohexylamino)-2-hydroxy-1-propanesulfonic acid (CAPSO) buffer, 2-(N-cyclohexylamino)ethanesulfonic acid (CHES) buffer, 3-[N,N-bis(2-hydroxyethyl)amino]-2-hydroxy-propanesulfonic acid (DIPSO) buffer, N-(2-hydroxyethylpiperazine)-N′-(3-propanesulfonic acid) (HEPPS) buffer, N-(2-hydroxyethyl)piperazine-N′-(2-hydroxypropancsulfonic acid) (HEPPSO) buffer, 2-(N-morpholine)ethanesulfonic acid (MES) buffer, triethanolamine buffer, imidazole buffer, glycine buffer, ethanolamine buffer, phosphate buffer, 3-(N-morpholine)-2-hydroxypropanesulfonic acid (MOPSO) buffer, piperazine-N,N′-bis(2-ethanesulfonic acid) (PIPES) buffer, piperazine-N,N′-bis(2-hydroxypropanesulfonic acid) (POPSO) buffer, N-tris[(hydroxymethyl)methyl]-3-aminopropanesulfonic acid (TAPS) buffer, 2-hydroxy-3-[tris(hydroxymethyl)methylamino]-1-propanesulfonic acid (TAPSO) buffer, N-[Tris(hydroxymethyl)methyl]-2-aminoethanesulfonic acid (TES) buffer, N-[Tris(hydroxymethyl)methyl]glycine (tricine) buffer, 2-amino-2-methyl-1,3-propanediol buffer, 2-amino-2-methyl-1-propanol buffer, and combinations thereof.

In some cases, a reagent can comprise a chelator. A chelator can be a compound that may bind available metals (e.g., Mg2+ and Ca2+) to such an extent that metals that remain available to the metal-dependent enzymes (e.g., deoxyribonucleases) are insufficient to support catalysis (i.e., nucleic acid degradation). For example, a metal independent enzyme may include a DNA ligase (e.g., D4 DNA ligase), a DNA polymerase (e.g., T7 DNA polymerase), an exonuclease (e.g., exonuclease 2, lamda-exonuclease), a kinase (e.g., T4 polynucleotide kinase), a phosphotase (e.g., BAP and CIP phosphotase), a nuclease (e.g., BL31 nuclease and XO nuclease), and an RNA-modifying enzyme (e.g., RNA polymerase, SP6, T7, T3 RNA polymerase, and T4 RNA ligase). In some cases, a chelator can be ethylenediaminetetraacetic acid (EDTA), [ethylenebis(oxyethylenenitrilo)]tetraacetic acid (EGTA) and 1,2-bis(2-aminophenoxy)ethane-N,N,N′,N′-tetraacetic acid (BAPTA), and/or salts thereof.

A chelator may be present at any desirable concentration. Where two or more chelators are included in a single reagent, either the concentration of each chelator or the total concentration of the combined chelators may fall within any of the provided ranges. In some embodiments, a chelator may include EDTA, EGTA, BAPTA, imidazole, iminodiacetate (IDA), bis(5-amidino-2-benzimidazolyl)methane (BABIM), and/or salts thereof.

In some embodiments, a chelator can have a final concentration of about 0.1 Molar to about 2 Molar. In some embodiments, a chelator can have a final concentration of at least about 0.001 Molar, at least about 0.005 Molar, at least about 0.01 Molar, at least about 0.05 Molar, at least about 0.1 Molar, at least about 0.5 Molar, or at least about 1 Molar. In some embodiments, a chelator can have a final concentration of not more than about 0.1 Molar, not more than about 0.5 Molar, not more than about 1 Molar, not more than about 2 Molar, or not more than about 3 Molar.

A buffer can comprise a metabolic modulator. A metabolic modulator can be a molecule that can act to optimize membrane penetration of chemistries within the reagent formulation, as well as stabilizing gene expression of cells, and in particular, hypoxic cancer cells. In some cases, a metabolic modulator can penetrate a tissue, a cell membrane, or an organelle membrane such as a nuclear membrane or a mitochondrial membrane. In some cases, a metabolic modulator can be polar aprotic solvents, DMSO, acetone, N,N-dimethylformanide, or acetonitrile. A metabolic modular can have a final concentration in the reagent of at least 0.1 Molar, at least 0.25 Molar, at least about 0.5 Molar, at least about 0.75 Molar. at least about 1.0 Molar, at least about 1.5 Molar, or at least 2.0 Molar A metabolic modulator can have a final concentration in the reagent of at most about 0.5 Molar, at most about 1.0 Molar, at most about 1.5 Molar, at most about 2.0 Molar, at most about 2.5 Molar, or at most about 3.0 Molar.

In some cases, a reagent can comprise an apoptosis substrate. An apoptosis substrate can be a compound or molecule which is a key component in the reduction of apoptosis. An apoptosis substrate works synergistically with other reagent formulation components to prevent apoptosis and foster cell stability and cell growth. In some embodiments, apoptosis can refer to the process of programmed cell death (PCD) that may occur in multicellular organisms. Biochemical events can lead to characteristic cell changes (morphology) and in some cases death. These changes can include blebbing, cell shrinkage, nuclear fragmentation, chromatin condensation, and chromosomal DNA fragmentation. In some cases, an apoptosis substrate can be a hormone. In some cases, an apoptosis substrate can be leptin.

An apoptosis substrate can have a final concentration in a reagent of at least 0.001 Molar, at least 0.01 Molar, at least 0.1 Molar, at least 0.5 Molar, at least 1 Molar, at least 1.5 Molar, or at least 2 Molar. An apoptosis substrate can have a final concentration in a reagent of not more than 0.1 Molar, not more than 0.5 Molar, not more than 1.0 Molar, not more than 1.5 Molar, not more than 2.0 Molar, not more than 3 Molar, not more than 4 Molar, or not more than 5 Molar.

In some embodiments, the cell viability reagent comprises polyvinyl sulfone, a chaotrope, at least one kosmotrope, a chelator, a buffer, a metabolic modulator, and an apoptosis substrate. These components of the reagent can be found in low molar concentrations, for example compared to other tissue preservatives.

Polyvinyl sulfone can be found for example in a concentration of between 0.1 Molar and 3.0 Molar. The polyvinyl sulfone concentration can be optimized to maximize biological sample stability, including stability of RNA within the biological sample, while minimizing toxic effects of the polyvinyl sulfone (e.g., toxic effects to the user).

Low molar concentrations of chaotropes can act differently compared with high molar concentrations of chaotropes, and can demonstrate a very protective effect on the stability and preservation of nucleic acids, as well as having a major impact on cell metabolism by modifying water distribution and metabolism of cells as described herein. Basis for U.S. Pat. No. 6,458,546 B1 (Baker).

The final concentration for the chaotrope can be from about 0.1 Molar to about 2 Molar. The final concentrations for the at least two kosmotropes can be from about 0.1 Molar to about 2 Molar for each kosmotrope. The final concentration for the chelator can be from about 0.1 Molar to about 2 Molar. The final concentration for the apoptosis substrate can be from about 0.001 Molar to about 0.5 Molar.

Unlike high molar concentrations of chaotropes which can destroy cells and denature proteins and nucleic acids, lower concentration of the chaotropes can be beneficial in preserving tissue samples. Also, low molar concentrations of kosmotropes can be synergistic with low concentrations of chaotropes, which can modulate cellular metabolism, in some cases protecting proteins and nucleic acids. In some cases, polyvinyl sulfone can act synergistically with a chaotrope, a kosmotrope, or a combination thereof to protect proteins and nucleic acids, including RNA. Optimum stabilization of biological macromolecules (e.g. cancer cells) requires a mixture of one or more of polyvinyl sulfone, kosmotropic anions and a chaotropic action.

Reagents herein can comprise one or more of: sodium thiocyanate, EDTA, DMSO, glycerol, sodium phosphate buffer(s), and α,α-trehalose. In some embodiments, reagents herein can comprise all of: EDTA, DMSO, glycerol, sodium phosphate buffer(s), and α,α-trehalose. In some embodiments, reagents herein can additionally comprise other components.

Reagents provided herein can comprise sodium thiocyanate. In some embodiments, sodium thiocyanate can inhibit exogenous and endogenous proteases responsible for destruction of a nucleic acid (RNA or DNA) sequence in a biological sample, such as a SARS-CoV-2 target RNA sequence. In some embodiments, sodium thiocyanate inhibits endogenous endonucleases and/or exonucleases that may degrade genetic material. Sodium Thiocyanate can combine with DMSO to produce an efficient pre-viral lysis step, and can allow for a more complete final sample lysis step. Use of a reagent comprising sodium thiocyanate can result in better target amplification than use of a same reagent not comprising sodium thiocyanate. In some embodiments, a reagent contains at least about 0.1%, about 0.5%, about 1%, about 1.5%, about 2%, about 2.5%, about 3%, about 5% or more sodium thiocyanate. In some embodiments, a reagent contains less than about 5%, about 4%, about 3%, about 2.5%, about 2%, about 1.5%, about 1%, about 0.5%, about 0.1% or less. In some embodiments, a reagent contains no sodium thiocyanate. In some embodiments, a reagent contains less than about 0.5% sodium thiocyanate. In some embodiments, a reagent contains less than about 1% sodium thiocyanate. In some embodiments, a reagent contains less than about 2% sodium thiocyanate. In some embodiments, a reagent contains less than about 3% sodium thiocyanate. As used herein, the percentages provided may be by volume (e.g., volume by volume), or by weight (e.g., weight by volume, weight by weight). In some embodiments, a percentage is provided as a molar percentage (i.e., mole fraction).

Reagents provided herein can comprise EDTA. EDTA can be used to bind metal ions, e.g., magnesium and/or calcium ions. EDTA can inhibit metalloproteases that can have a negative effect (direct or indirect) on a nucleic acid sequence, such as a nucleic acid target fragment. In some embodiments, EDTA enhances the stability of genetic information in a sample by inhibiting a metalloprotease's ability to degrade nucleic acid sequences. In some embodiments, EDTA is used in combination with one or more components disclosed herein to synergistically preserve genetic material in a sample.

Reagents provided herein can comprise dimethyl sulfoxide (DMSO). DMSO can drive cellular penetration or modulate cellular metabolic function (e.g., viral cellular metabolic function). In some embodiments, DMSO combined with EDTA in a reagent can drive cellular penetration or modulate cellular metabolic function (e.g., viral cellular metabolic function). In some embodiments, the concentration of DMSO in a reagent provided herein can be optimized to provide optimal results when a biological sample or nucleic acid thereof is subjected to PCR analysis. In alternative embodiments, a reagent may contain no DMSO. Reagents containing DMSO may comprise up to about 0.5%, about 1%, about 1.5%, about 2%, about 2.5%, about 3%, about 3.5%, about 4%, about 5%, about 6%, about 7%, about 8%, about 9%, about 10%, about 15%, about 20%, or more DMSO by volume. In some embodiments, a reagent contains less than about 20%, about 15%, about 10%, about 5%, about 4%, about 3.5%, about 3%, about 2.5%, about 2%, about 1.5%, about 1%, or less DMSO by volume. In some embodiments, a reagent contains about 1% to about 10% DMSO by volume. In some embodiments, a reagent contains about 1% to about 5% DMSO by volume. In some embodiments, a reagent contains about 1% to about 3% DMSO by volume. In some embodiments, a reagent contains about 2% to about 10% DMSO by volume. In some embodiments, a reagent contains about 2% to about 5% DMSO by volume. In some embodiments, a reagent contains about 2% to about 4%. In some embodiments, a reagent contains about 3% to about 10% DMSO by volume. In some embodiments, a reagent contains about 3% to about 5% DMSO by volume. In some embodiments, a reagent contains about 5% to about 10% DMSO by volume. In some embodiments, a reagent contains about 1% DMSO by volume. In some embodiments, a reagent contains about 2% DMSO by volume. In some embodiments, a reagent contains about 3% DMSO by volume. In some embodiments, a reagent contains about 4% DMSO by volume. In some embodiments, a reagent contains about 5% DMSO by volume. DMSO, as used herein, may be ACS reagent grade, biological grade, molecular biology grade, or the like. In some instances, DMSO is at least about 95%, about 97%, about 98%, about 99%, about 99.5% about 99.9% or greater in purity. In some embodiments, DMSO as used herein is substantially water free. In other embodiments, DMSO as used herein contains water. In some embodiments, contains one or more additional components (e.g., diluents, stabilizing agents, adjuvants, and the like). In some embodiments, DMSO as used herein optionally contains sodium thiocyanate.

Reagents provided herein can comprise glycerol. As used herein, glycerol or glycerin (alternatively glycerine) may be used interchangeably. Glycerol can be a kosmotrope that can stabilize cell contents against thermodynamic stress or protect cellular proteins against damage. In some embodiments, glycerol combined with α,α-trehalose in a reagent provided herein can stabilize cell contents against thermodynamic stress or protect cellular proteins against damage. In some embodiments, glycerol can add strength to a cellular component against environmental hypoxia. Reagents containing glycerol may comprise up to about 0.5%, about 1%, about 1.5%, about 2%, about 2.5%, about 3%, about 3.5%, about 4%, about 5%, about 6%, about 7%, about 8%, about 9%, about 10%, about 15%, about 20%, or more glycerol by volume. In some embodiments, a reagent contains less than about 20%, about 15%, about 10%, about 5%, about 4%, about 3.5%, about 3%, about 2.5%, about 2%, about 1.5%, about 1%, or less glycerol by volume. In some embodiments, a reagent contains about 1% to about 10% glycerol by volume. In some embodiments, a reagent contains about 1% to about 5% glycerol by volume. In some embodiments, a reagent contains about 1% to about 3% glycerol by volume. In some embodiments, a reagent contains about 2% to about 10% glycerol by volume. In some embodiments, a reagent contains about 2% to about 5% glycerol by volume. In some embodiments, a reagent contains about 2% to about 4%. In some embodiments, a reagent contains about 3% to about 10% glycerol by volume. In some embodiments, a reagent contains about 3% to about 5% glycerol by volume. In some embodiments, a reagent contains about 5% to about 10% glycerol by volume. In some embodiments, a reagent contains about 1% glycerol by volume. In some embodiments, a reagent contains about 2% glycerol by volume. In some embodiments, a reagent contains about 3% glycerol by volume. In some embodiments, a reagent contains about 4% glycerol by volume. In some embodiments, a reagent contains about 5% glycerol by volume. glycerol, as used herein, may be ACS reagent grade, biological grade, molecular biology grade, or the like. In some instances, glycerol is at least about 95%, about 97%, about 98%, about 99%, about 99.5% about 99.9% or greater in purity. In some embodiments, glycerol as used herein is substantially water-free. In some embodiments, glycerol as used herein contains water. In some embodiments, glycerol contains one or more additional components (e.g., diluents, stabilizing agents, or contaminants).

Reagents provided herein can comprise one or more sodium phosphate buffers. A sodium phosphate buffer can provide a biological pH system which can allow for optimal cellular metabolic attenuation by components of reagents herein. In alternative embodiments, a reagent can comprise one or more potassium phosphate buffers. In some embodiments, a buffer described herein (e.g., a phosphate buffer), provides a physiologically relevant osmolarity and/or ionic concentration so as to stabilize nucleic acid structures. In some embodiments, a combination of monobasic potassium phosphate (KH₂PO₄) and tribasic potassium phosphate (K₃PO₄) is used in a reagent described herein. In some embodiments, a combination of phosphate buffers provides a biological pH and ionic concentration that allows for optimal nucleic acid stabilization for RNA analysis. Any of the phosphate buffers described herein, (e.g., sodium, potassium, and the like) may be provided in a hydrated form. In some embodiments, a reagent contains a phosphate buffer that is sodium phosphate monobasic, sodium phosphate monobasic monohydrate, sodium phosphate monobasic dihydrate, sodium phosphate dibasic, sodium phosphate dibasic dihydrate, sodium phosphate tribasic, potassium phosphate monobasic, potassium phosphate dibasic, potassium phosphate tribasic, or a combination thereof. Phosphate buffers (or salts) may be provided in anhydrous or hydrated forms (e.g., monohydrate, dihydrate, hexahydrate, dodecahydrate, or any other commercially available form). In some embodiments, a reagent contains sodium phosphate. In some embodiments, a reagent contains potassium phosphate. In some embodiments, a reagent contains potassium phosphate dibasic salt, tribasic salt, or a combination thereof. In some instances, a reagent contains at least about 0.1%, about 0.5%, about 1%, about 1.5%, about 2%, about 2.5%, about 3%, about 5% or more phosphate buffer. A reagent may contain less than about 0.1%, about 0.5%, about 1%, about 1.5%, about 2%, about 2.5%, about 3%, or about 5% phosphate buffer. In some cases, a reagent contains less than about 0.5%, less than about 1%, less than about 1.5%, or less than about 2% phosphate buffer (e.g., NaH₂PO₄, Na₂HPO₄, Na₃PO₄, KH₂PO₄, K₂HPO₄, K₃PO₄).

Reagents provided herein can comprise α,α-trehalose. α,α-trehalose can be a kosmotrope that can protect cellular regulatory proteins and add stability to cells. In some embodiments, α,α-trehalose combined with glycerol can protect cellular regulatory proteins and add stability to cells. In some embodiments, α,α-trehalose combined with glycerol enhances the thermal stability of nucleic acid structures. In some embodiments, α,α-trehalose and glycerol synergistically enhance the stability of nucleic acid structures. In some embodiments, a combination of α,α-trehalose with glycerol prevents degradation, denaturation, and/or fragmentation of nucleic acid sequences or structures. In some embodiments, α,α-trehalose in a reagent herein can allow for increased cellular stabilization or reduced cellular autolytic activity, for example compared with a same reagent not comprising α,α-trehalose. In some embodiments, α,α-trehalose directly interacts with nucleic acids. In some embodiments, α,α-trehalose facilitates melting of double stranded DNA. In some embodiments, α,α-trehalose stabilizes single-stranded nucleic acids. In some embodiments, α,α-trehalose facilitates melting of double-stranded nucleic acids and stabilizes single-stranded nucleic acids, thus preserving the genetic material of a virus (e.g., ssRNA or ssDNA).

A reagent may contain α,α-trehalose in a hydrated form (e.g., α,α-trehalose dihydrate). As used herein, α,α-trehalose includes hydrated and anhydrous forms. In some embodiments, a reagent contains at least about 0.1%, about 0.5%, about 1%, about 1.5%, about 2%, about 2.5%, about 3%, about 5% or more α,α-trehalose. In some embodiments, a reagent contains less than about 5%, about 4%, about 3%, about 2.5%, about 2%, about 1.5%, about 1%, about 0.5%, about 0.1% or less α,α-trehalose. In some embodiments, a reagent contains no α,α-trehalose. In some embodiments, a reagent contains less than about 0.5% α,α-trehalose. In some embodiments, a reagent contains less than about 1% α,α-trehalose. In some embodiments, a reagent contains less than about 2% α,α-trehalose. In some embodiments, a reagent contains less than about 3% α,α-trehalose. As used herein, the percentages provided may be by volume (e.g., volume by volume), or by weight (e.g., weight by volume, weight by weight).

Reagents provided herein can comprise a viral inactivation viricide (alternatively, a viral inactivating agent, or a viricide). A viricide for use in a reagent described herein may be any detergent or surfactant capable of disrupting the lipid envelope or capsid layer of a virus or viral particle. In some embodiments, a viral inactivation viricide is an octylphenol ethoxylate (OPE) or a nonylphenol ethoxylate (NPE). In some embodiments, a viral inactivation viricide is 2-[4-(2,4,4-trimethylpentan-2-yl)phenoxy]ethanol. A viral inactivation viricide may comprise t-octylphenoxypolyethoxyethanol, polyethylene glycol tert-octylphenyl ether, Octylphenoxy poly(ethyleneoxy)ethanol (branched), 4-(1,1,3,3-Tetramethylbutyl)phenyl-polyethylene glycol, poly(oxy-1,2-ethanediyl)alpha[-4-(1,1,3,3-tetramethylbutyl)phenyl}-omega-hydroxy, Triton™ X, Triton™ X-45, Triton™ X-100, Triton™ X-102, Triton™ X-114, Triton™ X-165, Triton™ X-305, Triton™ X-405, Triton™ X-705, Octoxynol 9, Octoxinol 10, IGEPAL CA-720, IGEPAL CA-630, sodium dodecyl sulfate or sodium laurel sulfate (SDS), 3-[(3-cholamidopropyl)dimethylammonio]-1-propanesulfonate (CHAPS), cholic acid, polysorbate 20 (polyoxyethylene (20) sorbitan monolaurate, Tween 20), polysorbate 40 (polyoxyethylene (20) sorbitan monopalmitate, Tween 40), polysorbate 60 (polyoxyethylene (20) sorbitan monostearate, Tween 60), polysorbate 80 (polyoxyethylene (20) sorbitan monooleate, Tween 80), poloxamers and NP-40, or the like. In some embodiments, a viral inactivation viricide is a Triton™, ECOSURF™, or TERGITOL™ reagent. In some embodiments, a viral inactivation viricide is Triton™ reagent. In some embodiments, a viral inactivation viricide is Triton™ X, Triton™ X-45, Triton™ X-100, Triton™ X-102, Triton™ X-114, Triton™ X-165, Triton™ X-305, Triton™ X-405, Triton™ X-705, or the like. In some embodiments, a viral inactivation viricide is an ECOSURF™ reagent. In some embodiments, a viral inactivation viricide is ECOSURF™ EH-3, ECOSURF™ SA-4, ECOSURF™ SA-7, ECOSURF™ EH-6, ECOSURF™ EH-9, ECOSURF™ EH-9 (90%), ECOSURF™ SA-9, or the like. In some embodiments, a viral inactivation viricide is a TERGITOL™ reagent. In some embodiments, a viral inactivation viricide is TERGITOL™ NP-7, TERGITOL™ NP-9, TERGITOL™ NP-10, TERGITOL™ NP-40, TERGITOL™ TMN-3, TERGITOL™ TMN-6(90%), TERGITOL™ TMN-100X (90%), TERGITOL™ 15-S-3, TERGITOL™ 15-S-7, TERGITOL™ 15-S-9, TERGITOL™ 15-S-15, TERGITOL™ 15-S-20 (80%), TERGITOL™ 15-S-30, TERGITOL™ 15-S-40, TERGITOL™ 15-S-40 (70%), or the like.

In some embodiments, a viral inactivation viricide is Triton™ X. In other embodiments, the viral inactivation viricide is Triton™ X-100. In still other embodiments, a viral inactivation viricide is Triton™ X-305. A reagent containing a viral inactivation viricide may inactivate lipid-enveloped viruses or viral particles. In some embodiments, a viricide disrupts lipid membrane and/or protein coat of a virus, thus rendering the virus no longer infectious. In some embodiments, a viricide disrupts lipid membrane of SARS-CoV-2, rendering the coronavirus no longer infectious. In some embodiments, a virus is rendered no longer infectious within about 600 seconds (s), 500 s, 400 s, 300 s, 200 s, 100 s, 90 s, 80 s, 70 s, 60 s, 50 s, 40 s, 30 s, 20 s, 10 s, 9 s, 8 s, 7 s, 6 s, 5 s of contacting a reagent disclosed herein (e.g., one containing a viral inactivation viricide), or less. In some instances, a reagent as described herein comprises no more than about 10%, about 8%, about 6%, about 5%, about 4%, about 3%, about 2%, about 1%, about 0.5%, or less of a viral inactivation viricide by volume. In some embodiments, a reagent comprises at least about 0.5%, about 1%, about 1.5%, about 2%, about 2.5%, about 3%, about 3.5%, about 4%, about 5%, about 6%, about 8%, or about 10% or more of a viral inactivation viricide by volume. A reagent may, in some instances, contain less than about 1% of a viral inactivation viricide by volume. In some embodiments, a reagent contains less than about 2% of a viral inactivation viricide by volume. In some embodiments, a reagent contains less than about 3% of a viral inactivation viricide by volume. In some embodiments, a reagent contains less than about 5% of a viral inactivation viricide by volume. In some embodiments, a reagent contains less than about 10% of a viral inactivation viricide by volume. In some embodiments, a reagent herein does not contain a viral inactivation viricide.

Reagents provided herein may comprise a biocide. A “biocide” as used herein is any component that effectively controls (i.e., inhibits, inactivates, kills) microorganisms. Preferably, a biocide as used herein has broad spectrum antimicrobial activity with low toxicity to human subjects at the concentrations used. In some embodiments, a biocide inhibits the growth of bacteria, fungi (e.g., yeasts), and/or protozoa. In some embodiments, a biocide is non-toxic to mammals (e.g., humans). In some embodiments, a biocide disclosed herein provides antimicrobial activity for extended periods of time (i.e., does not degrade or become inactive for at least 5 days, 10 days, 15 days, 20 days, 28 days, 2 months, 6 months, 1 year, 2 years, or more). Examples of biocides include modified alkyl carboxylate mixtures of 5-Chloro-2-methyl-3(2H)-isothiazolone and 2-methyl-3(2H)-isothiazolone, Kathon 886, kathon biocide, CMIT/MIT. In some embodiments, a biocide comprises one or more substituted or unsubstituted isothiazolinones. In some embodiments, a biocide comprises 5-Chloro-2-methyl-3(2H)-isothiazolone (CMIT). In some embodiments, a biocide comprises 2-methyl-3(2H)-isothiazolone (MIT). In some embodiments, a biocide comprises a mixture of 5-Chloro-2-methyl-3(2H)-isothiazolone and 2-methyl-3(2H)-isothiazolone (CMIT/MIT). In some embodiments, a biocide is a ProClin™ composition comprising CMIT/MIT. In some embodiments, the biocide comprises an alkyl carboxylate stabilizer. The biocide may, in some instances, be ProClin™ 150, ProClin™ 200, ProClin™ 300, ProClin™ 950, and the like. In some embodiments, the biocide (e.g. CMIT/MIT or ProClin™ 300) is provided in a concentration of at least about 0.5%, 1%, 1.5%, 2%, 2.5%, 3%, 3.5%, 4%, 4.5%, 5%, 10%, or greater. A reagent as provided herein may contain a biocide provided in a concentration of about 10%, 5%, 4%, 3%, 1.5%, or less. A biocide for use in the present disclosure may comprise various salts (e.g., magnesium, copper, phosphate). In some embodiments, the biocide is salt-free. In some embodiments, a biocide reagent, as described herein, comprises no more than about 10%, 8%, 6%, 5%, 4%, 3%, 2%, 1%, 0.5%, or less of the total reagent composition. In some embodiments, a biocide reagent comprises at least about 0.5%, 15, 1.5%, 2%, 2.5%, 3%, 3.5%, 4%, 5%, 6%, 8%, or 10% or more of the total reagent composition. A reagent may comprise between about 1% and about 3% of a biocide by volume. A reagent may comprise between about 1.5% and about 2.5%. In some embodiments, a reagent provided herein comprises less than about 1% of a biocide. In some embodiments, a reagent provided herein comprises less than about 1.5% of a biocide. In some embodiments, a reagent provided herein comprises less than about 2% of a biocide. In some embodiments, a reagent provided herein comprises less than about 3% of a biocide.

In some embodiments, the biocide is ProClin™ 300. Reagents provided herein may comprise water. Water for use in the present disclosure may be ionized or deionized water. Water may be filtered, sterilized, distilled, or a combination thereof. In some instances, the water is sterilized. In some instances, the water is ACS reagent grade. In some instances, the water is Type I water or ultrapure water. In other instances, the water is Type II water. Examples of purified water include, by way of non-limiting example, Millipore water, OmniPur water, ACS reagent grade water, molecular biology grade water, and HPLC grade water. A reagent may comprise at least about 50%, about 60%, about 70%, about 75%, about 80%, about 85%, about 90%, about 95%, about 96%, about 97%, about 98%, or more water by volume. A reagent may comprise less than about 98%, about 97%, about 96%, about 95%, about 90%, about 85%, about 80%, about 75%, about 70%, about 65%, about 60%, or less water by volume. In some instances, a reagent comprises about 70% to about 90% water by volume. In some instances, a reagent comprises about 80% to about 90% water by volume. In some instances, a reagent comprises about 80% to about 85% water by volume. In some instances, a reagent comprises about 85% to about 90% water by volume. In some instances, water as used herein is of a known pH or is within a known pH range. In some embodiments, water contains a buffer. In some embodiments, water as used herein is at a pH of about 4, about 5, about 6, about 7, about 8, about 9, or about 10. In some embodiments, water as used herein is at a neutral pH or an approximately neutral pH (e.g., between about 6 and about 8, or between about 6.5 and about 7.5). In some instances, water is acidic. In some instances, water is alkaline or basic.

### Kits

Also provided herein are kits for preserving or stabilizing a biological sample. A kit can comprise a vessel configured to hold a reagent described herein and the biological sample. In some cases, a kit can additionally comprise means for collecting the biological sample.

The vessel can be a cup or tube of ample size to contain the biological sample and reagent. Examples of vessels can include but are not limited to an Eppendorf or similar tube, a test tube (in some cases with a cap, lid, or stopper), a cup, a beaker, a 6 well plate, a 12 well plate, a 24 well plate, or a 48 well plate a 96 well plate. In some embodiments, a kit can further comprise a lid, cap, or stopper for covering or sealing the vessel.

A kit can comprise a reagent as described herein comprising a sulfone such as polyvinyl sulfone. In some cases, the reagent can further comprise at least one chaotrope, at least one kosmotrope, a chelator, a buffer, an apoptosis substrate, a metabolic modulator, or a combination thereof. In some cases, a kit can comprise ingredients for making a reagent and instructions for mixing the ingredients to yield the reagent.

In some cases, a kit can comprise instructions for preserving or stabilizing a biological sample. The instructions can comprise instructions for contacting a biological sample with the reagent for example as described in the methods below.

### Methods of Use

Also contemplated herein are methods for preserving a biological sample or a biological molecule within a biological sample, and methods for stabilizing a biological sample or a biological molecule within a biological sample.

Methods can comprise contacting a biological sample with a reagent provided herein to yield a stabilized biological sample. The stabilized biological sample can comprise the biological molecule. In some embodiments, the reagent can comprise a first kosmotrope, a second kosmotrope, and a chaotrope. In some embodiments, the reagent can protect the biological molecule from degradation. In some embodiments, a component of the reagent can protect the biological molecule from degradation. In some embodiments, a combination of two or more components of the reagent can protect the biological molecule from degradation. In some embodiments, a reagent that protects the biological molecule or a viral particle from degradation is referred to as a viral transport medium or VTM. In some cases, a reagent that protects the biological molecule or viral particle from degradation is not a VTM.

In some embodiments, the biological sample can be of a subject. For example, the subject can be a human. In some embodiments, the biological sample can be of a subject suspected of being infected with a pathogen. In some embodiments, a biological sample is an upper respiratory tract sample, e.g., a pathogen that can be inactivated and/or preserved by reagents of the present disclosure include any virus or viral particle, for example, a lipid-enveloped virus. In some embodiments, the viral particle is inactivated by disrupting a lipid envelope while preserving the genetic information contained within (e.g., RNA or DNA). In some embodiments, the virus or viral particle is inactivated by disrupting or disassembling a virus or viral particle's protein coat or capsid. In some embodiments, reagents herein disrupt or disassemble a virus or viral particle's capsid and lipid envelope while preserving the genetic material disposed therein.

In some embodiments, a reagent herein achieves the goal of stabilizing genetic information (e.g., RNA) and inactivating the virus itself, thereby increasing safety while maintaining or enhancing the capacity to perform genetic analysis on a viral sample. In some embodiments, a reagent herein preserves genetic information (e.g., RNA) and rapidly inactivates a virus or viral sample within about 5 seconds of contacting the reagent. In some embodiments, a reagent herein preserves genetic information (e.g., RNA) and rapidly inactivates a virus or viral sample within about 10 seconds of contacting the reagent. In some embodiments, a reagent herein preserves genetic information (e.g., RNA) and rapidly inactivates a virus or viral sample within about 15 seconds of contacting the reagent. In some embodiments, a reagent herein preserves genetic information (e.g., RNA) and rapidly inactivates a virus or viral sample within about 20 seconds of contacting the reagent. In some embodiments, a reagent herein preserves genetic information (e.g., RNA) and rapidly inactivates a virus or viral sample within about 30 seconds of contacting the reagent.

In some embodiments, the pathogen can be an RNA virus. In some embodiments, the pathogen can be a coronavirus. In some embodiments, the pathogen can be SARS-CoV-2. In some embodiments, the pathogen can be SARS-CoV-2 or a variant thereof. In some embodiments, the pathogen can be a SARS-CoV-2 variant. By way of non-limiting example, a SARS-CoV-2 variant may be an alpha variant, beta variant, gamma variant, or delta variant. In some embodiments, the pathogen can be an influenza virus. In some embodiments, a pathogen can be an influenza A virus.

In some embodiments, a pathogen can be a H1N1 virus, H1N2 virus, H3N2 virus, H5N1 virus, and the like. In some embodiments, a pathogen can be a HIV virus. In some embodiments, a pathogen can be a viroid, a bacterium, a fungus, or a protozoan. In some embodiments, a pathogen can be an Adenovirus, Influenza A/B virus, rotavirus, rhinovirus, dengue virus, rabies virus, west Nile virus, vaccinia virus, human coronavirus n163 coronavirus, human coronavirus hku1 (hcov-hku1), yellow fever virus, human metapneumovirus, middle east respiratory syndrome (MERS) coronavirus (MERS-CoV).

A biological sample can comprise a sample of a component of a respiratory system, such as one provided herein. In some embodiments, for example, a sample can be obtained via a nasopharyngeal swab. In some embodiments, a sample can be obtained from saliva, blood, pre-ejaculate, semen, or vaginal fluids. In some embodiments, a biological sample comprises one or more cells, one or more fragments of cells, one or more viral particle, one or more viral particle fragment, one or more fungal particle, one or more fungal particle fragment, or any combination thereof. In some embodiments, a biological sample comprises a single cell (e.g., for single cell genomics), or one or more fragments thereof. In some embodiments, a biological sample comprises a single viral particle, or one or more fragments thereof. In some embodiments, a biological sample comprises a single fungal particle, or one or more fragments thereof. In some embodiments, a biological sample comprises a single bacterium, or one or more fragments thereof. In some embodiments, the biological sample comprises an isolated cell, or one or more fragments thereof. In some embodiments, the biological sample comprises a single patient cell. In some embodiments, a biological sample comprises a plurality of patient cells. In some embodiments, a biological sample comprises a plurality of viral particles and/or fragments thereof. In some embodiments, a biological sample comprises a plurality of fungal particles and/or fragments thereof. In some embodiments, a biological sample comprises a plurality of bacteria and/or fragments thereof. In some embodiments, the biological sample can comprise SARS-CoV-2. In some embodiments, the biological sample can comprise RNA of SARS-CoV-2. In some embodiments, the biological sample can comprise an RNA fragment of SARS-CoV-2.

A biological molecule of a biological sample can be a protein, a DNA molecule, or an RNA molecule. In some embodiments, a biological molecule of a biological sample can be another biological molecule. In some embodiments, a biological molecule that is RNA can be RNA of SARS-CoV-2. In some embodiments, a reagent described herein is used to stabilize pathogen samples. In some embodiments, a reagent as described herein is used to stabilize and/or preserve genetic material, e.g., RNA or DNA, from a pathogen sample that comprises cells, fragments of lysed cells, viral material, fungal material, and/or bacterial material.

In some embodiments, a reagent containing a viral inactivation viricide inactivates lipid-enveloped viruses or viral particles. In some embodiments, a reagent containing a viral inactivation viricide inactivates lipid-enveloped viruses or viral particles within about 60 seconds, within about 45 seconds, within about 30 seconds, within about 20 seconds, within about 15 seconds, within about 10 seconds, within about 5 seconds, or less after contacting the virus or viral particle, or a biological sample containing the same. In some embodiments, a reagent containing a viral inactivation viricide inactivates lipid-enveloped viruses or viral particles within about 30 seconds of contacting the virus or viral particle, or a biological sample containing the same. In some embodiments, a reagent containing a viral inactivation viricide inactivates lipid-enveloped viruses or viral particles within about 15 seconds of contacting the virus or viral particle, or a biological sample containing the same. In some embodiments, a reagent containing a viral inactivation viricide inactivates lipid-enveloped viruses or viral particles within about 10 seconds of contacting the virus or viral particle, or a biological sample containing the same. In some embodiments, a reagent containing a viral inactivation viricide inactivates lipid-enveloped viruses or viral particles within about 5 seconds of contacting the virus or viral particle, or a biological sample containing the same.

In some embodiments, a reagent containing a viral inactivation viricide inactivates lipid-enveloped viruses or viral particles without substantially lysing cells contained in the same sample. In some embodiments, a reagent containing a viral inactivation viricide inactivates lipid-enveloped viruses or viral particles within about 10 seconds of contacting the virus or viral particle without substantially lysing cells or disrupting proteins contained in the same sample. As used here, “substantially lysing cells” means less than about 25%, less than about 20%, less than about 15%, less than about 10%, less than about 5%, less than about 4%, less than about 3%, less than about 2%, less than about 1%, or fewer cells in a given sample undergo cell lysis. Preferably, less than about 10%, less than about 5%, or less than about 3% of cells undergo lysis in a given sample (e.g., between the time the sample is contacted with the reagent and when it is ultimately analyzed or processed).

In some embodiments, a reagent disclosed herein containing a viral inactivation viricide preserves (i.e., protects and/or stabilizes) nucleic acid sequences from (i) thermal degradation (e.g., from intermittent or prolonged exposure to high temperature, freeze/thaw cycles, hot/cold cycles); (ii) enzymatic degradation (i.e., inhibits metalloproteases, endonucleases, exonucleases, ribonucleases, and/or other enzymes with RNAse activity); and (iii) chemical degradation (i.e., by balancing pH and ion concentration/osmolarity); while reducing pathogenicity by rapidly (e.g., within about 10 seconds, about 15 seconds, or about 30 seconds) inactivating viruses or viral particles.

In some embodiments, a reagent disclosed herein protects nucleic acid sequences from (i) thermal degradation; (ii) enzymatic degradation; and (iii) chemical degradation. In some embodiments, a reagent disclosed herein provides greater safety (e.g., of a human subject handling pathogens) compared to a reagent that does not contain a viral inactivation viricide (e.g., a fixing agent such as formalin) by rapidly inactivating the pathogen.

In one aspect, provided herein is a method for preserving genetic information, for example, of a virus, virion, viroid, or viral particle. In some embodiments, the method comprising contacting a virus or virion, or a biological sample suspected of containing the same, with a reagent disclosed herein. In some embodiments, the method further comprises inactivating virus or virion, or a biological sample suspected of containing the same, with a reagent disclosed herein, thereby creating an inactivated viral sample.

In some embodiments, the inactivating takes place within about 5 seconds of contacting the virus or virion, or biological sample suspected of containing the same, with a reagent disclosed herein. In some embodiments, the inactivating takes place within about 10 seconds of contacting the virus or virion, or biological sample suspected of containing the same, with a reagent disclosed herein. In some embodiments, the inactivating takes place within about 15 seconds of contacting the virus or virion, or biological sample suspected of containing the same, with a reagent disclosed herein. In some embodiments, the inactivating takes place within about 20 seconds of contacting the virus or virion, or biological sample suspected of containing the same, with a reagent disclosed herein. In some embodiments, the inactivating takes place within about 30 seconds of contacting the virus or virion, or biological sample suspected of containing the same, with a reagent disclosed herein. In some embodiments, the inactivating takes place within about 60 seconds of contacting the virus or virion, or biological sample suspected of containing the same, with a reagent disclosed herein.

In some embodiments, the method further comprises storing and/or transporting the inactivated viral sample. In some embodiments, the storing and/or transporting the inactivated viral sample comprises exposing the inactivated viral sample to ambient and/or high temperature, as described herein.

In some embodiments, the storing and/or transporting comprises exposing the reagent, and the viral sample contained therein, to ambient and/or high temperatures for up to 7 days. In some embodiments, the storing and/or transporting comprises exposing the reagent, and the viral sample contained therein, to ambient and/or high temperatures for up to 14 days. In some embodiments, the storing and/or transporting comprises exposing the reagent, and the viral sample contained therein, to ambient and/or high temperatures for up to 21 days. In some embodiments, the storing and/or transporting comprises exposing the reagent, and the viral sample contained therein, to ambient and/or high temperatures for up to 28 days. In some embodiments, the storing and/or transporting comprises exposing the reagent, and the viral sample contained therein, to ambient and/or high temperatures for at least 7 days. In some embodiments, the storing and/or transporting comprises exposing the reagent, and the viral sample contained therein, to ambient and/or high temperatures for at least 14 days. In some embodiments, the storing and/or transporting comprises exposing the reagent, and the viral sample contained therein, to ambient and/or high temperatures for at least 21 days. In some embodiments, the storing and/or transporting comprises exposing the reagent, and the viral sample contained therein, to ambient and/or high temperatures for at least 28 days. In some embodiments, the storing and/or transporting the inactivated viral sample comprises exposing the inactivated viral sample to freeze-thaw and/or hot-cold cycles, as described herein.

In some embodiments, the genetic information (e.g., viral RNA) is preserved such that, subsequent to storage and/or transport, the genetic information is capable of being analyzed using standard genetic analysis procedures known in the art. In some embodiments, subsequent to storage and/or transport, the genetic information is capable of being sequenced, identified, quantified, or otherwise characterized using standard genetic analysis procedures known in the art. Examples of standard genetic analysis procedures known in the art include polymerase chain reaction (PCR) analysis (e.g., standard PCR, reverse transcriptase PCR, or real time PCR), northern blotting, in situ hybridization, expression microarrays, single-cell genomics, next-generation sequencing (NGS), or sequencing analysis (e.g., RNAseq). In some embodiments, genetic analysis procedures include isothermal nucleic acid amplification technology (INAAT), transcription mediated amplification, loop-mediated isothermal amplification (LAMP), CRISPR, mass spectrometry, single cell transcriptomics, or single cell sequencing.

In some embodiments, the method for preserving genetic information disclosed herein reduces the risk of infection to a human subject that handles, acquires, transports, analyzes, or is proximal to a biological sample suspected of containing a virus or virion. In some embodiments, the method for preserving genetic information disclosed herein reduces the risk of infection to a human subject that handles, acquires, transports, analyzes, or is proximal to an inactivated viral sample.

In another aspect, provided herein is a method of inactivating a pathogen (e.g., a virus or viral particle). In some embodiments, the inactivating comprises contacting the pathogen with a reagent disclosed herein. In some embodiments, the method of inactivating a pathogen comprises contacting a biological sample containing or suspected of containing the pathogen with a reagent (e.g., a reagent comprising a viral inactivation viricide or VTM) disclosed herein. In some embodiments, the method further comprises disrupting (e.g., denaturing, dissolving, disassembling) the lipid envelope of a virus or virion, or the protein coat (i.e., capsid) of a virus or virion, e.g., with a viral inactivation viricide. In some embodiments, the method further comprises protecting genetic material (e.g., nucleic acid sequences or structures) from degradation (e.g., enzymatic, thermal, or chemical degradation). In some embodiments, the method further comprises stabilizing single-stranded RNA, optionally coupled with melting double-stranded DNA. In some embodiments, the method further comprises inhibiting enzymes with RNAse activity (e.g., metalloproteases, endonucleases, exonucleases, ribonucleases, and the like). In some embodiments, the method further comprises chelating metal ions, thereby inhibiting metalloproteases. In some embodiments, the method further comprises stabilizing genetic material, for example, with a combination of a first kosmotrope (e.g., glycerol) and a second kosmotrope (e.g., α,α-trehalose).

In yet another aspect, provided herein is a method of storing and/or transporting a biological sample containing or suspected of containing SARS-CoV-2.

### EXAMPLES

In some embodiments, the first kosmotrope can be glycerol. In some embodiments, the second kosmotrope can be α,α-trehalose. In some embodiments, the chaotrope can be sodium thiocyanate. In some embodiments, a reagent can further comprise DMSO. In some embodiments, a reagent can comprise no DMSO. In some embodiments, a reagent does not comprise a fixing agent. In some embodiments, a reagent can comprise leptin or a variant thereof. In some embodiments, a reagent can comprise no leptin or a variant thereof. In some embodiments, a reagent can further comprise a buffer, such as a sodium phosphate buffer.

In some embodiments, a reagent of the present disclosure contains a first kosmotrope (e.g., glycerol), a second kosmotrope (e.g., α,α-trehalose), a chaotrope (e.g. sodium thiocyanate), and a buffer (e.g., potassium phosphate dibasic, tribasic, or a combination thereof). In some embodiments, a reagent further comprises a viral inactivation viricide (e.g., Triton™ X, Triton™ X-100), a biocide (e.g., ProClin™ 200 or ProClin™ 300), or a combination thereof. In some embodiments, a reagent contains a solvent or diluent such as DMSO, water, or a combination thereof. A sample reagent of the present disclosure, Reagent I, may comprise some or all of glycerol, α,α-trehalose, sodium thiocyanate, potassium phosphate dibasic, potassium phosphate tribasic, Triton™ X-100, ProClin™ 300, DMSO, and water. In some embodiments, a reagent, e.g., Reagent I-A comprises about 2% to about 5% glycerol, less than about 1% α,α-trehalose dihydrate, less than about 1% sodium thiocyanate, less than about 1% potassium phosphate dibasic, less than about 1% potassium phosphate tribasic, less than about 2% Triton™ X-100, less than about 5% ProClin™ 300, about 2% to about 5% DMSO, and about 80% to about 85% water.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Example 1

In an example, Reagent I-A is evaluated for its effectiveness in inactivating SARS-CoV-2 virus. For comparison, Reagent I-A is compared to Reference Device 1 (the DNA/RNA Shield Collection Tube, K202641).

SARS-CoV-2 (Isolate Japan/Ty-7-503/2021) was obtained from BEI Resources. This virus aliquot is used to create SARS-CoV-2 stocks for the inactivation studies using Reagent I-A. To establish virus stocks, African green monkey kidney cells are grown, and are then infected one day post-plating at ˜80% confluence. Cells are monitored for evidence of virus-induced cytopathic effect (CPE). When the culture showed ˜70% CPE at 48 hours post-infection, the cell culture media was harvested. The supernatant was stored in 0.5 mL aliquots at −80° C. An aliquot was tested by TCID₅₀ assay and found to be 4.73×10⁷ TCID₅₀/mL. PCR analysis using the CDC SARS-CoV-2 N1 assay showed the virus stock to be 1.8×10¹⁰ copies/mL.

Aliquots containing 20% virus stock, 5% nasal wash aspirate from clinically negative samples, and 75% Reagent I-A are provided in a collection tube at room temperature. Nasal aspirate and Reagent I-A are first provided in a sample tube at room temperature, then the virus stock is introduced and mixed thoroughly. After an amount of time specified below in Tables 1.1 and 1.2, the inactivation procedure is quenched and filtered by a Pierce Detergent Removal 0.5 mL (Lot: WC320233 MFG: Thermo Scientific) purification column follow the manufacturer's instructions. Samples are prepared and evaluated in 5 replicates. Purified viral samples are serially diluted 10-fold for 8 dilutions, which are then plated with Very E6 cells (10,000/well) and incubated for 4 days. At 4 days post-infection, microscopy analysis is performed to assess virus CPE within the cells.

TABLE 1
Summary of SARS-COV-2 Inactivation Titers in Reagent I-A vs. Control (PBS); n = 5

| Incubation Reagent | I-A | I-A | I-A | I-A | I-A | PBS |
|---|---|---|---|---|---|---|
| Duration | 10 sec | 30 sec | 1 min | 15 min | 30 min | 30 min |
| Average titer in TCID₅₀/mL | A | A | A | A | A | E |
| (SARS-CoV-2 Variant Japan/Ty-7-503/2021) | | | | | | |
| Viral Titer log reduction | >3 | >3 | >3 | >3 | >3 | 0 |
| (1 × 10^{x} TCID50) | | | | | | |

A: <50;
B: 50-500;
C: 500-5000;
D: 5000-50,000;
E: 50,000-500,000;
F: >500,000

Data in Table 1 show no detectable CPE after inactivation in the viral titer study for any of the time points evaluated using Reagent I-A, with a lower detection threshold of about 3.2E+01 TCID₅₀/mL. The control (PBS) showed substantial CPE. The viral titer log reduction is calculated by subtracting the average TCID₅₀ logarithm value of the control sample with PBS buffer by the average logarithm value of each treatment condition (Table 1). The log reduction in viral titer after incubated in Reagent I-A is at least greater than 3 at every time point evaluated. This virus inactivation performance of Reagent I-A exceeds the that of Reference Device 1 in SARS-CoV-2 inactivation, which reported >2.0 log reduction in viral titer after a 30 min exposure.

### Example 2

In another example, Reagent I-A is evaluated to determine the lowest detectable level of SARS-CoV-2 virus after spiking into Reagent I-A media and establish SARS-CoV-2 RNA stability in Reagent I-A.

### Preliminary Limit of Detection Analysis

Limit of detection testing is initially performed by spiking multiple concentrations of genomic equivalent copies (GEC) of SARS-CoV-2 into clinically negative matrices (from nares swab) with Reagent I-A in a collection sample tube, or Reference Device 2 (DNA/RNA Shield, Catalog #R1100-50, Zymo Research). Each sample matrix is prepared and tested individually.

Samples are spiked with inactivated SARS-CoV-2 (BEI catalog #NR-52286) at the following concentrations: 500 GEC/μL, 250 GEC/μL, 125 GEC/μL, 62.5 GEC/μL, 31.3 GEC/μL, 15.6 GEC/μL, 7.8 GEC/μL and 3.9 GEC/μL. The concentrations overlap with the detection range reported by the FDA authorized rt-PCR test used in the study. Three replicates are prepared for each concentration.

The SARS-CoV-2 RNA from samples containing varied SARS-CoV-2 GEC/μl are extracted using Mag-Bind Viral DNA/RNA 96-well kit (Omega BIO-TEK, Cat #M6246-03) on the Hamilton STAR automated extraction platform, following manufacturer's recommendations. After RNA extraction, real-time PCR is conducted using FDA authorized Logix Smart Coronavirus Disease 2019 (COVID-19) kit (Co-Diagnostics, Inc.) on the Applied Biosystems 7500 Fast Dx 96-well platform, following manufacturer's recommendations.

The acceptance criteria for determining the lowest detectable GEC for SARS-CoV-2 spiked into Reagent I-A is defined as when all three replicates for the concentration are detected by the qPCR assay.

TABLE 2
Summary of SARS-CoV-2 Preliminary Limit of Detection; n = 3

| SARS-CoV-2 conc. | Reagent I-A | Reference Device 2 |
|---|---|---|
| (GEC/μL) | Avg (Ct) | Avg (Ct) |
| 500 | A | A |
| 250 | A | A |
| 125 | A | A |
| 62.5 | A | B |
| 31.25 | B | B |
| 15.63 | B | B |
| 7.81 | B | D |
| 3.9 | D | D |

A: 30-33;
B: 34-36;
C: 36-40;
D: >40

Successful qPCR runs contain all positive and negative controls passing quality metrics. The data is represented as the average cycle threshold (Ct) values for 3 replicates (Table 2). Samples with Ct values >40 (D) are considered as undetermined by the instrument. For the initial limit of detection testing, the lowest SARS-CoV-2 viral concentration registering a qPCR Ct value <40 for all three replicates is defined as the limit of detection. Using the pre-defined data analysis plan, the initial limit of detection of the Reagent I-A samples and the Reference Device 2 samples are determined to be 7.81 GEC/μL and 15.63 GEC/μL, respectively.

Under similar conditions, Reagent I-A was evaluated for limit of detection in a side-by-side analysis with Reference Device 3 (the Center for Disease Control and Prevention's Viral Transport Medium (CDC VTM)). This analysis determined the limit of detection for Reagent I-A to be 7.81 GEC/μL while Reference Device 3 was detected (by the abovementioned conditions) at a concentration of 15.63 GEC/μL, similar to Reference Device 2. These data demonstrate a lower limit of detection for Reagent I-A when compared to Reference Device 2 and Reference Device 3.

### Confirmatory Limit of Detection Analysis

A confirmatory limit of detection analysis is performed with samples containing 7.81 GEC/μL SARS-COV-2 RNA in Reagent I-A. Each sample matrix is prepared and tested individually. An acceptance criterion for confirmatory limit of detection testing is used to determine the concentration that yielded at least 95% of the replicates recoverable at the concentration.

The confirmatory limit of detection analysis consists of an additional 30 real time qPCR replicates of the previously identified limit of detection for SARs-CoV-2 concentration at 7.81 GEC/μL. The percentage of recoverable samples in the 30 confirmatory replicates passing quality metrics is 100% (30/30), with an average Ct falling in the “B” range (34-36), consistent with the preliminary findings reported in Table 2.

Based on the pre-defined criteria, the confirmatory limit of detection analysis confirms a limit of detection of 7.81 GEC/μL, which is insubstantially lower than Reference Device 2 or Reference Device 3, which are tested in parallel. The limit of detection of 7.81 GEC/μL falls within the range of GEC/μL declared by the manufacture of FDA-authorized Logix Smart™ COVID-19 test kit for SARS-CoV-2 detection (4.29-9.35 GEC/μL).

### Example 3

In yet another example, Reagent I-A is evaluated to determine the time period that SARS-CoV-2 RNA from upper respiratory specimens is preserved and kept stable at ambient temperature in Reagent I-A.

Clinically negative sample specimens from nares swab are spiked with inactivated SARS-CoV-2 (BEI catalog #NR-52286) at 50 GEC/μL and incubated in 2 mL of Reagent I-A at ambient temperature (20-25° C.). Twenty replicates are prepared for each time point—the time points evaluated being 1 day, 2 days, 3 days, 4 days, 5 days, 6 days, 7 days, 15 days, 21 days, and 28 days. At each time point, the samples are processed for RNA extraction. Baseline samples at Day 0 are immediately processed for RNA extraction after sample preparation. RNA from samples containing SARS-CoV-2 are extracted as described in Example 2. Pre-defined acceptance criteria of (+/−) 3.0 Ct values from time zero is used to establish stability and preservation of nucleic acids (RNA from SARS-CoV-2 virus) as determined by real-time PCR, for 20-25° C. without loss of detection signal using statistical analysis. Successful real time qPCR runs contain all positive and negative controls passing quality metrics.

The data is represented as the difference in average Ct value between Day 0 and each incubation time point. The average Ct value at Day 0 was 34.0 (n=20).

TABLE 3
Summary of SARS-COV-2 RNA Stability
in Reagent I-A at 20-25° C.

| Days (25° C.) | 0 | 1 | 2 | 3 | 4 | 5 | 6 | 7 | 15 | 21 | 28 |
|---|---|---|---|---|---|---|---|---|---|---|---|
| Change in Avg Ct | — | A | A | A | A | A | A | A | C | A | B |
| from Day 0 | | | | | | | | | | | |

A: ±(<0.5) Ct;
B: ±(0.5-1.0) Ct;
C: ±(1.1-2.0) Ct;
D: ±(2.1-3.0) Ct;
E: ±(>3.0) Ct

The result of the Reagent I-A SARS-CoV-2 stability study confirmed that RNA stability in Reagent I-A meets the acceptance criteria of +/−3.0 Ct after 28 days at 20-25° C. storage. This study demonstrates equivalent performance of Reagent I-A with the Reference Device (DNA/RNA Shield Collection Tube, K202641).

### Computer Systems

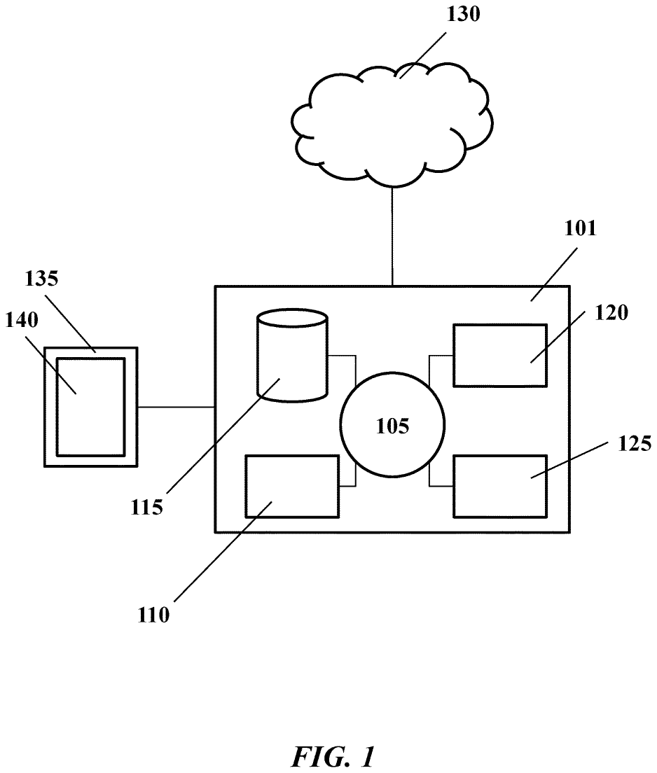

The present disclosure provides computer systems that are programmed to implement methods of the disclosure. FIG. 1 shows a computer system 101 that is programmed or otherwise configured to carry out methods of the present disclosure. The computer system 101 can regulate various aspects of the present disclosure. The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 105.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-65. (canceled)

66. A method for stabilizing a biological sample, the method comprising:
(a) providing a reagent comprising a kosmotrope, a chaotrope, and a viral-inactivating agent, said reagent configured to protect a biological molecule in said biological sample from destabilization or degradation; and
(b) contacting said biological sample comprising said biological molecule with said reagent to yield a stabilized biological sample comprising said biological molecule.

67. The method of claim 66, wherein said biological sample is obtained from a subject.

68. The method of claim 66, wherein said biological sample comprises a sample of a component of a respiratory system.

69. The method of claim 66, wherein said biological sample comprises a sample obtained via a nasopharyngeal swab.

70. The method of claim 66, wherein said biological molecule comprises a protein, a deoxyribonucleic acid molecule, a ribonucleic acid molecule, or any combination or variant thereof.

71. The method of claim 66, wherein said biological sample comprises SARS-CoV-2.

72. The method of claim 66, wherein said biological sample comprises a ribonucleic acid of SARS-CoV-2.

73. The method of claim 66, wherein said biological sample comprises a ribonucleic acid fragment of SARS-CoV-2.

74. The method of claim 66, wherein said kosmotrope comprises at least a first kosmotrope, and a second kosmotrope different from said first kosmotrope.

75. The method of claim 66, wherein said kosmotrope comprises glycerol, α,α-trehalose, or a combination thereof.

76. The method of claim 66, wherein said chaotrope is sodium thiocyanate.

77. The method of claim 66, wherein said reagent further comprises a chelator.

78. The method of claim 77, wherein said chelator is ethylenediaminetetraacetic acid.

79. The method of claim 66, wherein said reagent further comprises dimethylsulfoxide.

80. The method of claim 66, wherein said reagent further comprises a potassium phosphate buffer.

81. The method of claim 66, wherein said reagent further comprises a monobasic potassium phosphate buffer, a tribasic potassium phosphate buffer, or a combination thereof.

82. The method of claim 66, wherein said viral-inactivating agent is a viral inactivation viricide.

83. The method of claim 66, wherein said viral-inactivating agent comprises a solvent, a detergent, a surfactant, or any combination or variant thereof.

84. The method of claim 66, wherein said viral-inactivating agent comprises a nonionic surfactant.

85. The method of claim 66, wherein said reagent inactivates a pathogen within about 10 seconds without damaging genetic material of said pathogen.

86. The method of claim 66, wherein said reagent further comprises 5-Chloro-2-methyl-3(2H)-isothiazolone, 2-methyl-3(2H)-isothiazolone, or a combination thereof.

87. The method of claim 66, wherein said reagent enhances stability of genetic material to enzymatic degradation by a metalloprotease, endonuclease, exonuclease, ribonuclease, or a combination thereof.

88. A reagent for stabilizing a biological sample, comprising a kosmotrope, and a chaotrope for protecting a biological molecule in said biological sample from destabilization or degradation, and a viral-inactivating agent for inactivating a virus in said biological sample within 10 seconds.

89. The reagent of claim 88, further comprising one or more substituted or unsubstituted isothiazolinones.

90. The reagent of claim 88, further comprising an octylphenol ethoxylate or a nonylphenol ethoxylate.

91. The reagent of claim 88, wherein said biological molecule is a viral RNA.

92. The reagent of claim 88, wherein said biological molecule is SARS-CoV-2 RNA.
